(19) United States
(12) Patent Application Publication
(10) Pub. No.: US 2015/0148662 A1
(43) Pub. Date: May 28, 2015

(54) Technique for Determining Blood Velocity in a Blood Vessel

(71) Applicant: Julie Alex, Bangalore (IN)

(72) Inventor: Julie Alex, Bangalore (IN)

(21) Appl. No.: 14/550,605

(22) Filed: Nov. 21, 2014

(30) Foreign Application Priority Data
Nov. 22, 2013 (IN) 1322/KOL/2013

(51) Int. Cl. A61B 5/026; G06T 7/00; G01R 33/563; A61B 6/00; A61B 5/055

(52) U.S. Cl. CPC A61B 5/026; A61B 6/481; A61B 5/055; G01R 33/5635; G06T 7/0016
USPC 600/420; 600/431

(57) ABSTRACT

A method and a system for determining a blood velocity between a first point and a second point in a blood vessel are presented. In the method, a time series of registered images of the blood vessel are received and subsequently scanned to select a base image and a test image. The base image corresponds to a begin time that represents a time instance when a contrast agent reached the first point. The test image corresponds to an end time that represents a time instance when the contrast agent reached the second point. A time elapsed between the begin time and the end time is determined. A length of the blood vessel is determined. The blood velocity is calculated by comparing the length of the blood vessel and the elapsed time.

This application claims the benefit of IN 1322/KOL/2013, filed on Nov. 22, 2013, which is hereby incorporated by reference in its entirety.

### BACKGROUND

The present embodiments relate to the determination of blood flow.

Alteration of blood flow in blood vessels is associated with many medical conditions. One of the physiologic parameters of interest to determine blood flow is blood velocity in, for example, the blood vessels such as veins, arteries, and capillaries. For example, a measurement of the blood velocity through the heart and major vessels of the body may help physicians identify problems in heart valve performance, arterial stenosis, occlusions, aneurysms, tumors, atherosclerosis and other anomalies that may manifest in blood flow irregularities.

The distance blood flows inside the blood vessel in a given time period is the blood velocity. The blood velocity may be measured as centimeters/second (cm/s). The blood velocity differs for different blood vessels, and also for different regions of the same blood vessel. For example, the velocity of blood under normal physiological conditions in the aorta is approximately 30 cm/s, in arterioles, 1.5 cm/s, in capillaries, 0.04 cm/s, in venules, 0.5 cm/s, and in the vena cava, 8 cm/s.

To measure the blood velocity, conventionally, an ultrasonic Doppler blood flow velocity detection apparatus and catheter based blood velocity detection apparatuses are used. However, the conventional techniques are complicated and use expensive instrumentation. For catheter based blood velocity detection apparatuses, a catheter is positioned inside the body, and the apparatuses are thus are very inconvenient and complicated.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a simple technique for determining blood velocity in a blood vessel is provided.

According to a first aspect, a method for determining a blood velocity between a first point in a lumen of a blood vessel and a second point in the lumen of the blood vessel is provided. A contrast agent flows in the lumen of the blood vessel in a direction from the first point towards the second point. The method includes receiving a time series of registered images of a region of the blood vessel, scanning the time series of registered images, determining a time elapsed, determining a length of the blood vessel, and calculating the blood velocity. The region includes at least the first point and the second point.

In the scanning of the time series of registered images, a base image from the time series of registered images is selected. Similarly, a test image from the time series of registered images is selected. The base image corresponds to a begin time, and the test image corresponds to an end time. The begin time represents a time instance when the contrast agent reached the first point in the lumen of the blood vessel, and the end time represents a time instance when the contrast agent reached the second point in the lumen of the blood vessel. Subsequently, a time elapsed between the begin time and the end time is determined in the act of determining the time elapsed.

In the determining of the length of the blood vessel, a distance along the blood vessel between the first point and the second point is determined. In the calculating of the blood velocity, the blood velocity is calculated by comparing the length of the blood vessel and the elapsed time.

In an embodiment of the method, in the determining of the length of the blood vessel, a number of pixels of the test image representing the contrast agent between the first point and the second point is counted. In the method, a number of pixels representing an average width of the lumen of the blood vessel in the test image between the first and the second point are determined. A length value is determined by comparing the number of pixels of the test image representing the contrast agent between the first point and the second point and the number of pixels representing the average width of the lumen of the blood vessel between the first point and the second point. The length value represents the length of the blood vessel. This provides a simple embodiment of the method for determining the length of the blood vessel and uses only one image (e.g., the test image).

In another embodiment of the method, in the determining of the length of the blood vessel, a test image first edge count is determined. The test image first edge count is determined by counting a number of pixels forming a first edge of the lumen of the blood vessel between the first point and the second point in the test image. The test image first edge count represents the length of the blood vessel. This provides another alternative and simple embodiment of the method for determining the length of the blood vessel and uses only one image (e.g., the test image).

In another embodiment of the method, in the determining of the length of the blood vessel, a test image first edge count and a test image second edge count are determined. The test image first edge count is determined by counting a number of pixels forming a first edge of the lumen of the blood vessel between the first point and the second point in the test image. The test image second edge count is determined by counting a number of pixels forming a second edge of the lumen of the blood vessel between the first point and the second point in the test image. Subsequently, a test image average edge count is calculated. The test image average edge count is an average of the test image first edge count and the test image second edge count. The test image average edge count represents the length of the blood vessel. This provides another alternative and simple embodiment of the method for determining the length of the blood vessel and uses only one image (e.g., the test image). Since both the edges (e.g., the first edge and the second edge) are used in this embodiment, the accuracy of the method is improved compared to the embodiment that utilizes only the first edge.

In another embodiment of the method, in the determining of the length of the blood vessel, a topological skeleton of the lumen of the blood vessel between the first point and the second point is determined in the test image. Subsequently, a topological count of the test image is determined by counting pixels of the test image forming the topological skeleton of the lumen of the blood vessel in the test image. The topological count of the test image represents the length of the blood vessel. This provides an alternative and simple method for determining the length of the blood vessel and uses only one image (e.g., the test image). The accuracy of the method in this embodiment is high, as the topological skeleton represents a line drawn through the center of the lumen along the blood vessel.

In another embodiment of the method, in the determining of the length of the blood vessel, a number of pixels representing the contrast agent in the base image are counted. Similarly, and independent of the foregoing act, a number of pixels representing the contrast agent in the test image is counted. Subsequently, a net pixel-difference between the base image and the test image is determined. The net pixel-difference is a difference between the number of pixels representing the contrast agent in the base image and the number of pixels representing the contrast agent in the test image. In the method, a number of pixels in the test image representing an average width of the lumen of the blood vessel in the test image between the first point and the second point are determined. The net pixel-difference and the number of pixels representing the average width of the lumen of the blood vessel between the first point and the second point are compared to get a length value. The length value represents the length of the blood vessel. This provides a simple embodiment of the method for determining the length of the blood vessel and uses both images (e.g., the test image and the base image).

In another embodiment of the method, in the determining of the length of the blood vessel, a test image first edge count is determined. The test image first edge count is determined by counting a number of pixels forming a first edge of the lumen of the blood vessel in the test image. Similarly and independent of the foregoing act, a base image first edge count is determined. The base image first edge count is determined by counting a number of pixels forming a first edge of the lumen of the blood vessel in the base image. The first edge of the base image corresponds to the first edge of the test image. A first edge pixel-difference is determined. The first edge pixel-difference is a difference between the test image first edge count and the base image first edge count. The first edge pixel-difference represents the length of the blood vessel. This provides an alternative and simple embodiment of the method for determining the length of the blood vessel and uses both images (e.g., the test image and the base image).

In another embodiment of the method, in the determining of the length of the blood vessel, a test image first edge count and a test image second edge count are determined. The test image first edge count is determined by counting a number of pixels forming a first edge of the lumen of the blood vessel in the test image. The test image second edge count is determined by counting a number of pixels forming a second edge of the lumen of the blood vessel in the test image. Similarly and independent from the foregoing act, a base image first edge count and a base image second edge count are determined. The base image first edge count is determined by counting a number of pixels forming a first edge of the lumen of the blood vessel in the base image. The base image second edge count is determined by counting a number of pixels forming a second edge of the lumen of the blood vessel in the base image. The first edge of the base image corresponds to the first edge of the test image, and the second edge of the base image corresponds to the second edge of the test image. Subsequently, a first edge pixel-difference is determined. The first edge pixel-difference is a difference between the test image first edge count and the base image first edge count. Similarly, a second edge pixel-difference is determined. The second edge pixel-difference is a difference between the test image second edge count and the base image second edge count. An average edge pixel-difference is calculated. The average edge pixel difference is an average of the first edge pixel-difference and the second edge pixel-difference. The average edge pixel-difference represents the length of the blood vessel. This provides an alternative and simple embodiment of the method for determining the length of the blood vessel and uses both images (e.g., the test image and the base image). Moreover, since both the edges (e.g., the first edge and the second edge) of the test image as well as the base image are used in this embodiment, the accuracy of the method is improved compared to the embodiment that utilizes only the first edges of the test image and the base image.

In another embodiment of the method, in the determining of the length of the blood vessel, a topological skeleton of the lumen of the blood vessel in the test image is determined. Subsequently, a topological count of the test image is determined by counting pixels of the test image forming the topological skeleton of the lumen of the blood vessel in the test image. Similarly and independent from the foregoing act, a topological skeleton of the lumen of the blood vessel in the base image is determined. Subsequently, a topological count of the base image is determined by counting pixels of the base image forming the topological skeleton of the lumen of the blood vessel in the base image. A topological pixel-difference between the base image and the test image is determined. The topological pixel-difference is a difference between the topological count of the test image and the topological count of the base image. The topological pixel-difference represents the length of the blood vessel. This provides an alternative and simple embodiment of the method for determining the length of the blood vessel and using both images (e.g., the test image and the base image).

In another embodiment of the method, the registered images of the region of the blood vessel are acquired by using an X-ray based medical imaging technique. Thus, images acquired by any X-ray based medical imaging technique may be used to determine the blood velocity.

In another embodiment of the method, the X-ray based medical imaging technique is a digital subtraction angiography (DSA) technique. Since DSA is a simple and pervasively used technique of X-ray based medical imaging, the method of one or more of the present embodiments is simple to implement.

In another embodiment of the method, the registered images of the region of the blood vessel are acquired by using a magnetic resonance angiography technique. Thus, images acquired by any magnetic resonance angiography technique may be used to determine the blood velocity.

According to a second aspect, a system for determining a blood velocity between a first point in a lumen of a blood vessel and a second point in the lumen of the blood vessel is provided. A contrast agent flows in the lumen of the blood vessel in a direction from the first point towards the second point. The system includes an image acquisition module, an image registration module, and a processor. The image acquisition module is adapted to acquire a time series of images of a region of the blood vessel. The region includes at least the first point and the second point. The image registration module is adapted to register the time series of images acquired by the image acquisition module. The processor is adapted to perform a method as described in accordance with the first aspect of the present embodiments.

In an embodiment of the system, the image acquisition module is an X-ray based medical imaging device. Since X-ray based medical imaging devices are simple to use, cost effective and readily available, the system is simple to manufacture, cost effective and easy to handle.

In another embodiment of the system, the image acquisition module is a magnetic resonance based medical imaging device. Since magnetic resonance based medical imaging devices are simple to use, cost effective and readily available, the system is simple to manufacture, cost effective and easy to handle.

In accordance with a third aspect, a computer-readable medium having computer-executable instructions for performing the method of one or more of the present embodiments is presented.

### BRIEF DESCRIPTION OF THE DRAWINGS

FIG. 1 is a schematic representation of an exemplary blood vessel;

FIG. 2 is a flow chart illustrating one embodiment of a method including determining a length of the blood vessel;

FIG. 3 is a schematic representation of an exemplary embodiment of a time series of registered images including a test image and a base image;

FIG. 4 is a flow chart illustrating an embodiment of determining the length of the blood vessel using the test image;

FIG. 5 is a schematic representation of an exemplary embodiment of the test image;

FIG. 6 is a schematic representation of an exemplary embodiment of the base image;

FIG. 7 is a flow chart illustrating an embodiment of determining the length of the blood vessel using a test image first edge count;

FIG. 8 is a flow chart illustrating an embodiment of determining the length of the blood vessel using a test image first average edge count;

FIG. 9 is a flow chart illustrating an embodiment of determining the length of the blood vessel using a topological count of the test image;

FIG. 10 is a flow chart illustrating an embodiment of determining the length of the blood vessel using the test image and the base image;

FIG. 11 is a flow chart illustrating an embodiment of determining the length of the blood vessel using a first edge pixel-difference;

FIG. 12 is a flow chart illustrating an embodiment of determining the length of the blood vessel using an average edge pixel-difference;

FIG. 13 is a flow chart illustrating an embodiment of determining the length of the blood vessel using a topological pixel-difference; and

FIG. 14 is a schematic representation of one embodiment of a system.

### DETAILED DESCRIPTION

Hereinafter, above-mentioned and other features of the present technique are described in details. Various embodiments are described with reference to the drawings, where like reference numerals are used to refer to like elements throughout. In the following description, for purpose of explanation, numerous specific details are set forth in order to provide a thorough understanding of one or more embodiments. The illustrated embodiments are intended to explain, and not to limit the invention. Such embodiments may be practiced without these specific details.

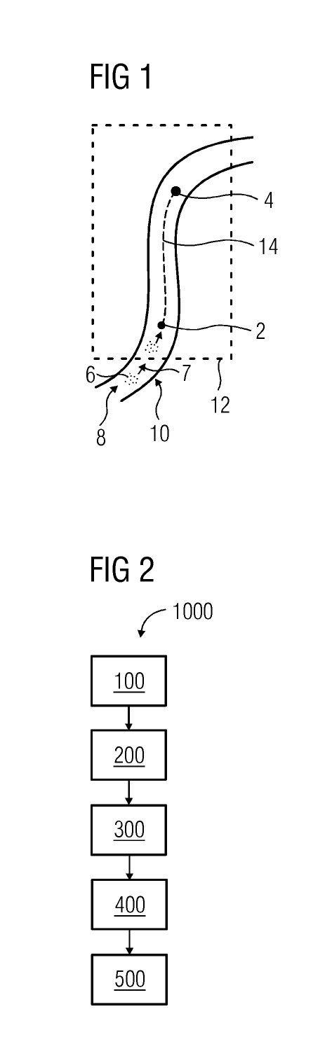

A blood velocity in a blood carrying vessel such as arteries, veins, capillaries and so forth is determined. FIG. 1 is a schematic representation of an exemplary blood vessel 10. The blood vessel 10 may be a tubular structure having walls (not shown) surrounding a lumen 8. The blood (not shown) flows in the lumen 8 of the blood vessel 10.

In accordance with aspects of the present embodiments, the blood velocity is determined between a first point 2 in the lumen 8 of the blood vessel 10 and a second point 4 in the lumen 8 of the blood vessel 10. The present technique is applied to the blood vessel 10 to which a contrast agent 6 is added. The addition of the contrast agents 6 such as, but not limited to, iodine based contrast mediums, gadolinium based contrast mediums, and so forth may be performed for various medical imaging techniques such as, but not limited to, X-ray imaging techniques, magnetic resonance angiography, and so forth. The contrast agent 6, when added to the blood, flows in the lumen 8 along with the blood, in a direction 7 (e.g., from the first point 2 towards the second point 4, in a flow direction of the blood).

In one embodiment, the blood velocity between the first point 2 and the second point 4 is determined from a distance 14 between the first point 2 and the second point 4 and a time taken to travel the distance 14.

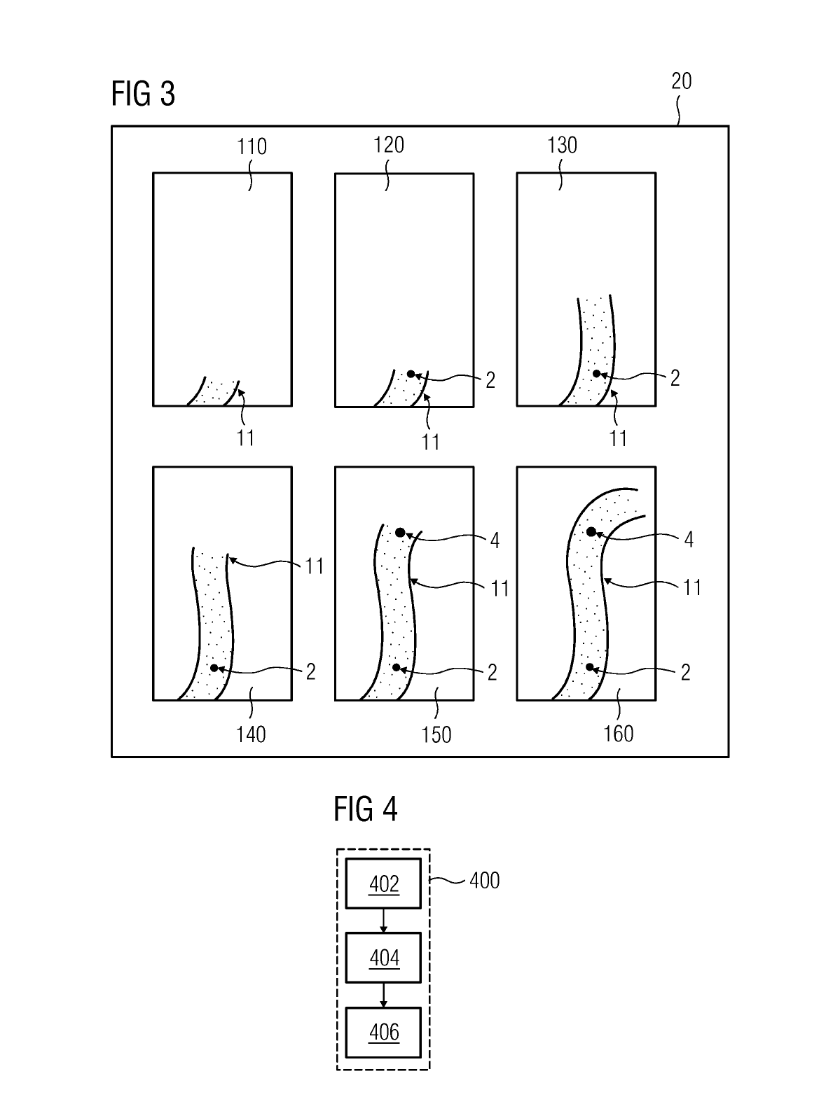

Referring to FIG. 1 in combination with FIGS. 2 and 3, a method 1000 is explained. FIG. 2 is a flow chart illustrating one embodiment of the method 1000. In the method 1000, the blood velocity between the first point 2 and the second point 4 is determined.

In the method 1000, a time series 20 of registered images 110, 120, 130, 140, 150, 160 of a region 12 of the blood vessel 10 is received in act 100. The region 12 includes at least the first point 2 and the second point 4. In one embodiment of the method 1000, the images 110, 120, 130, 140, 150, 160 of the region 12 of the blood vessel 10 may be acquired by using an X-Ray based medical imaging technique. The X-Ray based medical imaging technique may be, but not limited to, digital subtraction angiography (DSA) technique. In another embodiment of the method 1000, the images 110, 120, 130, 140, 150, 160 of the region 12 of the blood vessel 10 may be acquired by using a magnetic resonance angiography (MRA) technique.

FIG. 3 is a schematic representation of an exemplary embodiment of the time series 20 including the registered images 110, 120, 130, 140, 150, 160. The time series 20 is a sequence of registered images 110, 120, 130, 140, 150, 160, where the images 110, 120, 130, 140, 150, 160 are acquired at successive points in time. The successive points in time may be spaced at uniform time intervals. Thus, if the registered image 110 is acquired at a time ‘t’, then, for example, the registered image 120 is acquired after an interval ‘n’ from the time ‘t’ (e.g., at time ‘t+n’), and the registered image 130 is acquired after another interval ‘n’ from the time ‘t+n’ (e.g., at time ‘t+2n’), and the registered image 140 is acquired after another interval ‘n’ from time ‘t+2n’ (e.g., at time ‘t+3n’), and so on. Thus, the contrast agent 6 is visualized at different stages of propagation in the lumen 8 of the blood vessel 10 at the successive points in time.

In act 200 of the method 1000, the time series 20, as received in the step 100, is scanned, and a base image 120 and a test image 150 are selected from the registered images 110, 120, 130, 140, 150, 160 of the time series 20. The base image 120 corresponds to a begin time, and the test image 150 corresponds to an end time. As shown in FIG. 3, the begin time represents a time instance when the contrast agent 6 reached the first point 2 in the lumen 8 of the blood vessel 10, and the end time represents a time instance when the contrast agent 6 reached the second point 4 in the lumen 8 of the blood vessel 10.

Subsequently, in act 300 of the method 1000, a time elapsed is determined. The time elapsed between the begin time and the end time is determined by comparing the begin time and the end time corresponding to the base image 120 and the test image 150, respectively, and as selected in act 200. The time elapsed represents a time taken by the contrast agent 6 to flow from the first point 2 to the second point 4 in the lumen 8 of the blood vessel 10.

In act 400, independent from act 300, a length of the blood vessel 10 is determined. The length of the blood vessel 10 is the distance 14 between the first point 2 and the second point 4 measured along the blood vessel 10.

In act 500 of the method 1000, the blood velocity is calculated. The blood velocity is calculated by comparing the length of the blood vessel 10 as determined in act 400 and the elapsed time as determined in act 300.

The information about the begin time and the end time from which the time elapsed is determined in act 300 may be obtained from a time stamp of the base image 120 and the test image 150. Such time stamps may be assigned or associated with medical images when the medical images are acquired. Various embodiments of act 400 (e.g., the of determining of the length of the blood vessel 10) are explained hereinafter.

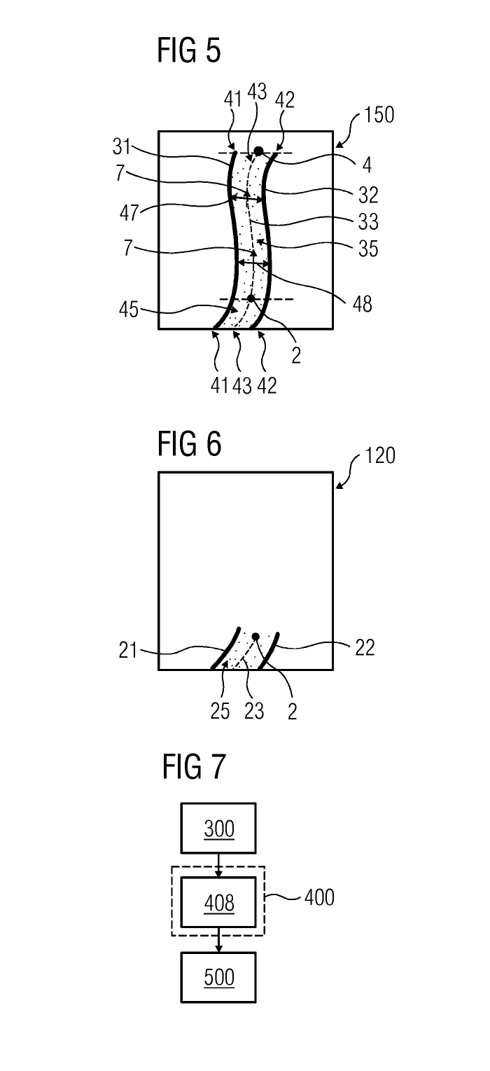

The different embodiment of act 400 are further explained in FIGS. 4, 7, 8, 9, 10, 11, 12, and 13 with the help of FIG. 5 and FIG. 6, along with FIGS. 1 and 3. FIG. 5 is a schematic representation of an exemplary embodiment of the test image 150 as selected in act 200, in accordance with the method 1000, from the time series 20, as depicted in FIG. 3. FIG. 6 is a schematic representation of an exemplary embodiment of the base image 120 as selected in act 200, in accordance with the method 1000, from the time series 20, as depicted in FIG. 3.

FIG. 4 is a flow chart depicting an embodiment of the act 400 of determining the length of the blood vessel 10 using the test image 150. In accordance with this embodiment of the method 1000, in act 400, a number of pixels of the test image 150 representing the contrast agent 6 between the first point 2 and the second point 4 is counted in act 402. Thus, in act 402, the pixels present in an area 35 in the test image 150 are counted. The area 35 in the test image 150 represents an area in the test image 150 in which the pixels represent a presence of the contrast agent 6 in portions of the region 12.

Independent of act 402, in the method 1000, a number of pixels representing an average width of the lumen 8 of the blood vessel 10 in the test image 150 between the first point 2 and the second point 4 is determined in act 404. The average width of the lumen 8 may be determined by calculating widths of the lumen 8 at different positions 47, 48 along the direction 7. The width is measured in a direction (not shown) perpendicular to the direction 7.

A length value is determined in act 406 by comparing the number of pixels of the test image 150 representing the contrast agent 6 between the first point 2 and the second point 4 as counted in act 402 and the number of pixels representing the average width of the lumen 8 between the first point 2 and the second point 4 as determined in act 404. The comparison as mentioned in act 406 is performed by dividing the number of pixels of the test image 150 representing the contrast agent 6 between the first point 2 and the second point 4 by the number of pixels representing the average width of the lumen 8 between the first point 2 and the second point 4.

In this embodiment of the method 1000, the length value as determined in the step 406 represents the length of the blood vessel 10. Dimensions of the pixel (e.g., length of each side of one pixel of the test image 150) are known, and the same may be used to obtain the length of the blood vessel 10. For example, when the pixels are square in shape, a side of the pixel will represent a length of the pixel. Thus, the length value is multiplied with the length of the pixel of the test image 150, and the length of the blood vessel 10 is obtained.

FIG. 7 is a flow chart illustrating another embodiment of act 400 of determining the length of the blood vessel 10. According to this embodiment, in act 400 of the method 1000, a test image first edge count is determined in act 408. The test image first edge count is determined in act 408 by counting a number of pixels forming a first edge 31 of the lumen 8 of the blood vessel 10 between the first point 2 and the second point 4 in the test image 150. The test image first edge count as determined in act 408 represents the length of the blood vessel 10. Similar to the foregoing embodiment, as described in reference to FIG. 4, the dimensions of the pixels for the test image 150 are used to determine the length of the blood vessel 10.

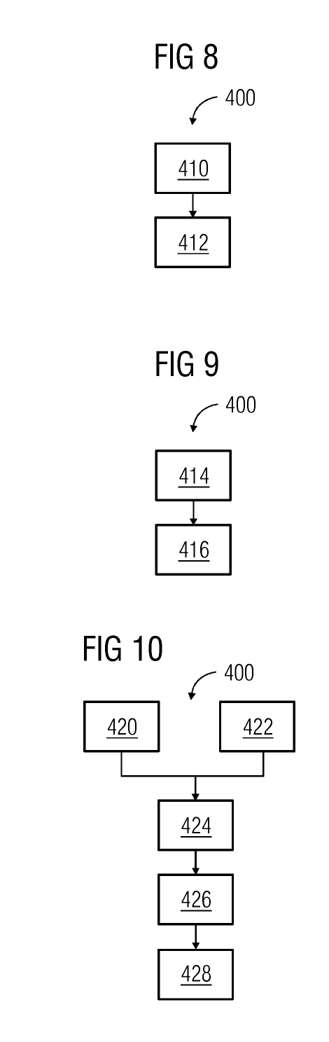

FIG. 8 is a flow chart illustrating yet another embodiment of act 400 of the method 1000. In accordance with this embodiment of the method 1000, a test image first edge count and a test image second edge count are determined in act 410. The test image first edge count is determined by counting a number of pixels forming the first edge 31 of the lumen 8 of the blood vessel 10 between the first point 2 and the second point 4 in the test image 150. Additionally, in act 410, the test image second edge count is determined by counting a number of pixels forming a second edge 32 of the lumen 8 of the blood vessel 10 between the first point 2 and the second point 4 in the test image 150.

Subsequently, a test image average edge count is calculated in act 412. The test image average edge count is an average of the test image first edge count and the test image second edge count as determined in act 410.

In this embodiment, the test image average edge count represents the length of the blood vessel 10. Similar to the foregoing embodiment, as described with reference to FIG. 4, the dimensions of the pixels for the test image 150 are used to determine the length of the blood vessel 10.

FIG. 9 is a flow chart illustrating yet another embodiment of act 400 of the method 1000. In accordance with this embodiment of the method 1000, a topological count of the test image 150 is determined. In the method 1000, the test image 150 is used, and in the test image 150 a topological skeleton 33 of the lumen 8 of the blood vessel 10 between the first point 2 and the second point 4 is determined in act 414.

The topological skeleton (e.g., skeleton) of a shape is a thin version of that shape that is equidistant to boundaries of the shape. The determining of such skeletons may be referred to as skeletonization. The skeletonization is well known and widely used in the field of shape analysis, and thus, the same has not been described herein for sake of brevity.

Subsequently, from the topological skeleton 33 so determined in the test image 150, a topological count of the test image 150 is determined in act 416. In act 416, the pixels of the test image 150 forming the topological skeleton 33 are counted. In this embodiment of the method 1000, the topological count of the test image 150 represents the length of the blood vessel 10. Similar to the foregoing embodiment, as described with reference to FIG. 4, the dimensions of the pixels for the test image 150 are used to determine the length of the blood vessel 10.

FIG. 10 is a flow chart illustrating yet another embodiment of act 400 of the method 1000. In accordance with this embodiment of the method 1000, a length value is determined. This embodiment uses both the test image 150 and the base image 120 for determination of the length value.

In this embodiment of the method 1000, a number of pixels representing the contrast agent 6 in the base image 120 are counted in act 420. This is done by counting the pixels in an area 25 of the base image 120, as shown in FIG. 6. The area 25 in the base image 120 represents an area in the base image 120 in which the pixels represent a presence of the contrast agent 6 in portions of the region 12.

Similarly, and independent of act 420, a number of pixels representing the contrast agent 6 in the test image 150 is counted in act 422. This is done by counting the pixels in an area 45 of the test image 150 as shown in FIG. 5. The area 45 in the test image 150 represents an area in the test image 150 in which the pixels represent a presence of the contrast agent 6 in portions of the region 12.

Subsequently, a net pixel-difference between the base image 120 and the test image 150 is determined in act 424. The net pixel-difference is a difference between the number of pixels representing the contrast agent 6 in the base image 120 as counted in act 420 and the number of pixels representing the contrast agent 6 in the test image 150 as counted in act 422.

Independent from act 420, 422 and 424, the number of pixels in the test image 150 representing the average width of the lumen 8 of the blood vessel 10 in the test image 150 between the first point 2 and the second point 4 is determined in act 426. The number of pixels in the test image 150 representing the average width of the lumen 8 of the blood vessel 10 is determined, as described with reference to FIG. 4.

The net pixel-difference, as determined in act 424, and the number of pixels representing the average width of the lumen 8 of the blood vessel 10 between the first point 2 and the second point 4, as determined in act 426, are compared to get a length value in act 428.

In this embodiment of the method 1000, the length value represents the length of the blood vessel 10. Similar to the foregoing embodiment, as described with reference to FIG. 4, the dimensions of the pixels for the base image 120 and the test image 150 are used to determine the length of the blood vessel 10. The dimensions of the pixels in the registered images 110, 120, 130, 140, 150, 160 and thus in the base image 120 and the test image 150 are equal to each other.

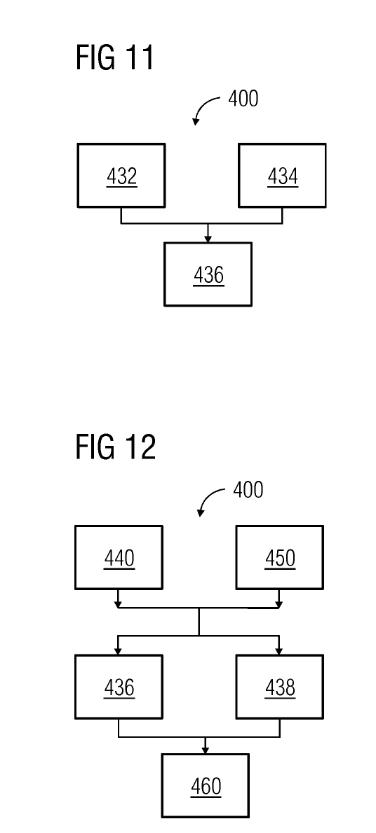

FIG. 11 is a flow chart illustrating yet another embodiment of act 400 of the method 1000. In accordance with this embodiment of the method 1000, a first edge pixel-difference is determined. This embodiment uses both the test image 150 and the base image 120 for determination of the first edge pixel-difference.

In this embodiment of the method 1000, a test image first edge count is determined in act 432. The test image first edge count is determined by counting a number of pixels forming a first edge 41 of the lumen 8 of the blood vessel 10 in the test image 150. Similarly, and independent from act 432, a base image first edge count is determined in act 434. The base image first edge count is determined by counting a number of pixels forming the first edge 21 of the lumen 8 of the blood vessel 10 in the base image 120.

Since the base image 120 and the test image 150 both represent the region 12 and since the base image 120 and the test image 150 are registered, the first edge 21 of the base image 120 corresponds to the first edge 41 of the test image 150.

A first edge pixel-difference is determined in act 436. The first edge pixel-difference is a difference between the test image first edge count, as determined in act 432, and the base image first edge count, as determined in act 434.

In this embodiment of the method 1000, the first edge pixel-difference represents the length of the blood vessel 10. Similar to the foregoing embodiments, as described with reference to FIGS. 4 and 10, the dimensions of the pixels for the base image 120 and the test image 150 are used to determine the length of the blood vessel 10.

FIG. 12 is a flow chart depicting yet another embodiment of act 400 of the method 1000. In accordance with this embodiment of the method 1000, an average edge pixel-difference is determined. This embodiment uses both the test image 150 and the base image 120 for determination of the average edge pixel-difference.

In this embodiment of the method 1000, the test image first edge count and a test image second edge count are determined in act 440. The test image first edge count is determined by counting the number of pixels forming the first edge 41 of the lumen 8 of the blood vessel 10 in the test image 150. The test image second edge count is determined by counting a number of pixels forming a second edge 42 of the lumen 8 of the blood vessel 10 in the test image 150.

Similarly, and independent from act 440, the base image first edge count and a base image second edge count are determined in act 450. The base image first edge count is determined by counting the number of pixels forming the first edge 21 of the lumen 8 of the blood vessel 10 in the base image 120. The base image second edge count is determined by counting a number of pixels forming a second edge 22 of the lumen 8 of the blood vessel 10 in the base image 120.

As the base image 120 and the test image 150 both represent the region 12 and are registered images, the first edge 21 of the base image 120 corresponds to the first edge 41 of the test image 150. The second edge 22 of the base image 120 corresponds to the second edge 42 of the test image 150.

A first edge pixel-difference is determined in act 436. The first edge pixel-difference is a difference between the test image first edge count as determined in act 440 and the base image first edge count as determined in act 450. Similarly, a second edge pixel-difference is determined in act 438. The second edge pixel-difference is a difference between the test image second edge count, as determined in act 440, and the base image second edge count, as determined in act 450.

An average edge pixel-difference is calculated in act 460. The average edge pixel difference is an average of the first edge pixel-difference, as determined in act 436, and the second edge pixel-difference, as determined in the step 438.

In this embodiment of the method 1000, the average edge pixel-difference represents the length of the blood vessel 10. Similar to the foregoing embodiment, as described in reference to FIGS. 4 and 10, the dimensions of the pixels for the base image 120 and the test image 150 are used to determine the length of the blood vessel 10.

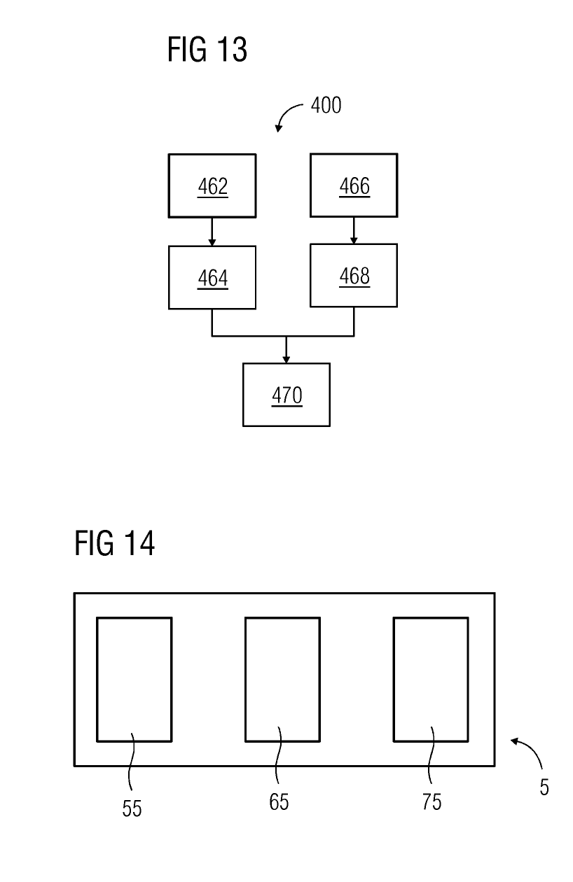

FIG. 13 is a flow chart illustrating yet another embodiment of act 400 of the method 1000. In accordance with this embodiment of the method 1000, a topological pixel-difference is determined. This embodiment uses both the test image 150 and the base image 120 for determination of the topological pixel-difference.

In this embodiment of the method 1000, a topological skeleton 43 of the lumen 8 of the blood vessel 10 in the test image 150 is determined in act 462. Subsequently and by using the topological skeleton 43, as determined in the step 462, a topological count of the test image 150 is determined in act 464. The topological count of the test image 150 is determined by counting pixels of the test image 150 that form the topological skeleton 43 of the lumen 8 of the blood vessel 10 in the test image 150.

Similarly and independent from acts 462 and 464, a topological skeleton 23 of the lumen 8 of the blood vessel 10 in the base image 120 is determined in act 466. Subsequently and by using the topological skeleton 23, as determined in act 466, a topological count of the base image 120 is determined in act 468. The topological count of the base image 120 is determined by counting pixels of the base image 120 that form the topological skeleton 23 in the base image 120.

A topological pixel-difference between the base image 120 and the test image 150 is determined in act 470. The topological pixel-difference is a difference between the topological count of the test image 150, as determined in act 464, and the topological count of the base image 120, as determined in act 468.

In this embodiment of the method 1000, the topological pixel-difference represents the length of the blood vessel 10. Similar to the foregoing embodiment described with reference to FIGS. 4 and 10, the dimensions of the pixels for the base image 120 and the test image 150 are used to determine the length of the blood vessel 10.

In accordance with aspects of the present embodiments, besides the method 1000, a system 5, as depicted in FIG. 14, is also presented.

In combination with FIGS. 1 to 13, FIG. 14 is used hereinafter to explain the system 5 for determining the blood velocity between the first point 2 in the lumen 8 of the blood vessel 10 and the second point 4 in the lumen 8 of the blood vessel 10. For application of the system 5, the contrast agent 6 is used in accordance with aspects of the present embodiments. As discussed with reference to FIG. 1, the contrast agent 6 flows in the lumen 8 of the blood vessel 10 in the direction 7.

The system 5 includes an image acquisition module 55, an image registration module 65, and a processor 75. The image acquisition module 55 is configured to acquire the time series 20 of images (not shown) of the region 12 of the blood vessel 10. The image acquisition module may be, but not limited to, an X-Ray based medical imaging device, a magnetic resonance based medical imaging device, or another imaging device. Such X-ray based medical imaging devices and magnetic resonance based medical imaging devices are well known in the art of medical imaging, and thus, the same has not been described herein for sake of brevity.

The image registration module 65 is configured to register the time series 20 of images acquired by the image acquisition module 55. As a result of registration of the images by the image registration module 65, the time series 20 of registered images 110, 120, 130, 140, 150, 160, as depicted in FIG. 3, is obtained. The image registration module 65 may be a computer processing unit including image registration algorithms. The construction and working of such modules for registration of images and such image registration algorithms are well known in the art of image analysis and compilation, and thus, the same has not been described herein for sake of brevity.

The processor 75 is configured to perform the method 1000, as described in accordance with the aspects of the present embodiments with reference to FIGS. 1 to 13.

The system 5 includes a display unit (not shown). The display unit is connected to the processor 75 and is adapted to display a representation of the blood velocity between the first point 2 in the lumen 8 of the blood vessel 10 and the second point 4 in the lumen 8 of the blood vessel 10. The representation of the blood velocity may be in form of, but not limited to, a combination of numerals, a combination of colors, a combination of symbols, a combination of alphabets, or another combination.

In an exemplary embodiment, the system 5 includes a memory (not shown) configured to store a reference dataset of blood velocities. Elements of the reference dataset of blood velocities represent a normal range of blood velocity for a given blood vessel under given physiological parameters. The given blood vessels may be, but not limited to, aorta, capillaries, arterioles, and/or other vessels. An example of the normal range of blood velocity may be 28 cm/s to 32 cm/s in the aorta. The physiological parameters may be age, gender, weight, medical condition of an individual, and/or other parameters. The processor 75 is further adapted to compare the blood velocity between the first point 2 in the lumen 8 of the blood vessel 10 and the second point 4 in the lumen 8 of the blood vessel 10 with the reference data set of blood velocities.

In accordance with aspects of the present embodiments, besides the method 1000 and the system 5, a computer-readable medium (e.g., a non-transitory computer-readable storage medium; not shown) having computer-executable instructions for performing the method 1000, as described with reference to FIGS. 1 to 13, is also presented.

A computer-readable medium (e.g., a computer-usable medium) is any apparatus that may contain, store, communicate, propagate, or transport the computer-executable instructions (e.g., computer programs) for use by or in connection with the system 5 and/or to perform the method 1000. The computer-readable medium is a physical computer-readable medium such as an electronic, magnetic, optical, electromagnetic, infrared, or semiconductor system (or apparatus or device), and/or another physical computer-readable medium. Examples of the physical computer-readable medium include a semiconductor or solid state memory, magnetic tape, a removable computer diskette, a random access memory (RAM), a read-only memory (ROM), a rigid magnetic disk and an optical disk such as compact disk-read only memory (CD-ROM), compact disk-read/write (CD-R/W) and DVD.

While the present technique has been described in detail with reference to certain embodiments, the present technique is not limited to the embodiments discussed. Rather, in view of the present disclosure, which describes exemplary modes for practicing the invention, many modifications and variations may be provided by those skilled in the art without departing from the scope and spirit of this invention. The scope of the invention is, therefore, indicated by the following claims rather than by the foregoing description. All changes, modifications, and variations coming within the meaning and range of equivalency of the claims are to be considered within the scope of the claims.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for determining a blood velocity between a first point in a lumen of a blood vessel and a second point in the lumen of the blood vessel, a contrast agent flowing in the lumen of the blood vessel in a direction from the first point towards the second point, the method comprising:
receiving a time series of registered images of a region of the blood vessel, wherein the region comprises at least the first point and the second point;
scanning the time series of the registered images to select a base image and a test image from the time series of the registered images, the base image corresponding to a begin time, and the test image corresponding to an end time, wherein the begin time represents a time instance when the contrast agent reached the first point in the lumen of the blood vessel, and the end time represents a time instance when the contrast agent reached the second point in the lumen of the blood vessel;
determining a time elapsed between the begin time and the end time;
determining a length of the blood vessel, wherein the length of the blood vessel is a distance along the blood vessel between the first point and the second point; and
calculating the blood velocity, the calculating comprising comparing the length of the blood vessel and the elapsed time.

2. The method of claim 1, wherein determining the length of the blood vessel comprises:
counting a number of pixels of the test image representing the contrast agent between the first point and the second point;
determining a number of pixels representing an average width of the lumen of the blood vessel in the test image between the first point and the second point; and
determining a length value, the determining of the length value comprising comparing the number of pixels of the test image representing the contrast agent between the first point and the second point, and the number of pixels representing the average width of the lumen of the blood vessel between the first point and the second point,
wherein the length value represents the length of the blood vessel.

3. The method of claim 1, wherein determining the length of the blood vessel comprises determining a test image first edge count, the determining of the test image first edge count comprising counting a number of pixels forming a first edge of the lumen of the blood vessel between the first point and the second point in the test image, and
wherein the test image first edge count represents the length of the blood vessel.

4. The method of claim 1, wherein determining the length of the blood vessel comprises:
determining a test image first edge count and a test image second edge count, the determining of the test image first edge count comprising counting a number of pixels forming a first edge of the lumen of the blood vessel between the first point and the second point in the test image, the determining of the test image second edge count comprising counting a number of pixels forming a second edge of the lumen of the blood vessel between the first point and the second point in the test image; and
calculating a test image average edge count, wherein the test image average edge count is an average of the test image first edge count and the test image second edge count, and wherein the test image average edge count represents the length of the blood vessel.

5. The method of claim 1, wherein the determining of the length of the blood vessel comprises:
determining a topological skeleton of the lumen of the blood vessel between the first point and the second point in the test image; and
determining a topological count of the test image, the determining of the topological count comprising counting pixels of the test image forming the topological skeleton of the lumen of the blood vessel in the test image,
wherein the topological count of the test image represents the length of the blood vessel.

6. The method of claim 1, wherein determining the length of the blood vessel comprises:
counting a number of pixels representing the contrast agent in the base image;
counting a number of pixels representing the contrast agent in the test image;
determining a net pixel-difference between the base image and the test image, wherein the net pixel-difference is a difference between the number of pixels representing the contrast agent in the base image and the number of pixels representing the contrast agent in the test image;
determining a number of pixels in the test image representing an average width of the lumen of the blood vessel in the test image between the first point and the second point; and
comparing the net pixel-difference and the number of pixels representing the average width of the lumen of the blood vessel between the first point and the second point to get a length value, wherein the length value represents the length of the blood vessel.

7. The method of claim 1, wherein determining the length of the blood vessel comprises:
determining a test image first edge count, the determining of the test image first edge count comprising counting a number of pixels forming a first edge of the lumen of the blood vessel in the test image;
determining a base image first edge count, the determining of the base image first edge count comprising counting a number of pixels forming a first edge of the lumen of the blood vessel in the base image, wherein the first edge of the base image corresponds to the first edge of the test image; and
determining a first edge pixel-difference, wherein the first edge pixel-difference is a difference between the test image first edge count and the base image first edge count, and wherein the first edge pixel-difference represents the length of the blood vessel.

8. The method of claim 1, wherein determining the length of the blood vessel comprises:
determining a test image first edge count and a test image second edge count, the determining of the test image first edge count comprising counting a number of pixels forming a first edge of the lumen of the blood vessel in the test image, the determining of the test image second edge count comprising counting a number of pixels forming a second edge of the lumen of the blood vessel in the test image;
determining a base image first edge count and a base image second edge count, the determining of the base image first edge count comprising counting a number of pixels forming a first edge of the lumen of the blood vessel in the base image, the determining of the base image second edge count comprising counting a number of pixels forming a second edge of the lumen of the blood vessel in the base image, wherein the first edge of the base image corresponds to the first edge of the test image, and the second edge of the base image corresponds to the second edge of the test image;
determining a first edge pixel-difference, wherein the first edge pixel-difference is a difference between the test image first edge count and the base image first edge count;
determining a second edge pixel-difference, wherein the second edge pixel-difference is a difference between the test image second edge count and the base image second edge count; and
calculating an average edge pixel-difference, wherein the average edge pixel-difference is an average of the first edge pixel-difference and the second edge pixel-difference, and wherein the average edge pixel-difference represents the length of the blood vessel.

9. The method of claim 1, wherein determining the length of the blood vessel comprises:
determining a topological skeleton of the lumen of the blood vessel in the test image;
determining a topological count of the test image, the determining of the topological count comprising counting pixels of the test image forming the topological skeleton of the lumen of the blood vessel in the test image;
determining a topological skeleton of the lumen of the blood vessel in the base image;
determining a topological count of the base image, the determining of the topological count comprising counting pixels of the base image forming the topological skeleton of the lumen of the blood vessel in the base image; and
determining a topological pixel-difference between the base image and the test image, wherein the topological pixel-difference is a difference between the topological count of the test image and the topological count of the base image, and wherein the topological pixel-difference represents the length of the blood vessel.

10. The method of claim 1, wherein the time series of registered images of the region of the blood vessel are acquired by an X-ray based medical imaging technique.

11. The method of claim 10, wherein the X-ray based medical imaging technique is a digital subtraction angiography technique.

12. The method of claim 1, wherein the registered images of the region of the blood vessel are acquired using magnetic resonance angiography technique.

13. A system for determining a blood velocity between a first point in a lumen of a blood vessel and a second point in the lumen of the blood vessel, a contrast agent flowing in the lumen of the blood vessel in a direction from the first point towards the second point, the system comprising:
an image acquisition scanner configured to acquire a time series of images of a region of the blood vessel, wherein the region comprises at least the first point and the second point;
an image registration module configured to register the time series of images acquired by the image acquisition module; and
a processor configured to:
scan the time series of the registered images to select a base image and a test image from the time series of the registered images, the base image corresponding to a begin time, and the test image corresponding to an end time, wherein the begin time represents a time instance when the contrast agent reached the first point in the lumen of the blood vessel, and the end time represents a time instance when the contrast agent reached the second point in the lumen of the blood vessel;
determine a time elapsed between the begin time and the end time;
determine a length of the blood vessel, wherein the length of the blood vessel is a distance along the blood vessel between the first point and the second point; and
calculate the blood velocity, the calculation comprising a comparison of the length of the blood vessel and the elapsed time.

14. The system of claim 13, wherein the image acquisition module is an X-ray based medical imaging device.

15. The system of claim 13, wherein the image acquisition module is a magnetic resonance based medical imaging device.

16. A non-transitory computer-readable storage medium having computer-executable instructions executable by one or more processors for determining a blood velocity between a first point in a lumen of a blood vessel and a second point in the lumen of the blood vessel, a contrast agent flowing in the lumen of the blood vessel in a direction from the first point towards the second point, the instructions comprising:
receiving a time series of registered images of a region of the blood vessel, wherein the region comprises at least the first point and the second point;
scanning the time series of the registered images to select a base image and a test image from the time series of the registered images, the base image corresponding to a begin time, and the test image corresponding to an end time, wherein the begin time represents a time instance when the contrast agent reached the first point in the lumen of the blood vessel, and the end time represents a time instance when the contrast agent reached the second point in the lumen of the blood vessel;
determining a time elapsed between the begin time and the end time;
determining a length of the blood vessel, wherein the length of the blood vessel is a distance along the blood vessel between the first point and the second point;
calculating the blood velocity, the calculating comprising comparing the length of the blood vessel and the elapsed time; and
calculating the blood velocity, the calculating comprising comparing the length of the blood vessel and the elapsed time.

17. The non-transitory computer-readable storage medium of claim 16, wherein determining the length of the blood vessel comprises:
counting a number of pixels of the test image representing the contrast agent between the first point and the second point;
determining a number of pixels representing an average width of the lumen of the blood vessel in the test image between the first point and the second point; and
determining a length value, the determining of the length value comprising comparing the number of pixels of the test image representing the contrast agent between the first point and the second point, and the number of pixels representing the average width of the lumen of the blood vessel between the first point and the second point,
wherein the length value represents the length of the blood vessel.

18. The non-transitory computer-readable storage medium of claim 16, wherein determining the length of the blood vessel comprises determining a test image first edge count, the determining of the test image first edge count comprising counting a number of pixels forming a first edge of the lumen of the blood vessel between the first point and the second point in the test image, and
wherein the test image first edge count represents the length of the blood vessel.

19. The non-transitory computer-readable storage medium of claim 16, wherein determining the length of the blood vessel comprises:
determining a test image first edge count and a test image second edge count, the determining of the test image first edge count comprising counting a number of pixels forming a first edge of the lumen of the blood vessel between the first point and the second point in the test image, the determining of the test image second edge count comprising counting a number of pixels forming a second edge of the lumen of the blood vessel between the first point and the second point in the test image; and
calculating a test image average edge count, wherein the test image average edge count is an average of the test image first edge count and the test image second edge count, and wherein the test image average edge count represents the length of the blood vessel.

20. The non-transitory computer-readable storage medium of claim 16, wherein the determining of the length of the blood vessel comprises:
determining a topological skeleton of the lumen of the blood vessel between the first point and the second point in the test image; and
determining a topological count of the test image, the determining of the topological count comprising counting pixels of the test image forming the topological skeleton of the lumen of the blood vessel in the test image,
wherein the topological count of the test image represents the length of the blood vessel.
